(19) United States
(12) Patent Application Publication
(10) Pub. No.: US 2012/0172775 A1
(43) Pub. Date: Jul. 5, 2012

(54) Wrist protector

(75) Inventors: Hsiu-Yuan Nien, New Taipei City (TW); Jung-Jung Chen, New Taipei City (TW)

(73) Assignee: EKTOUCH CO., LTD., New Taipei City (TW)

(21) Appl. No.: 13/064,931

(22) Filed: Apr. 27, 2011

(30) Foreign Application Priority Data
Dec. 31, 2010 (TW) 099225646

(51) Int. Cl. A61F 5/00

(52) U.S. Cl. 602/21

(57) ABSTRACT

A wrist protector includes a protector body having a flexible base member defining therein an accommodation chamber and a fastening structure adapted for fastening the flexible base member to a user's wrist, and a hard support plate detachably set in the accommodation chamber of the flexible base member to give support to the user's wrist. The hard support plate is orthopedically engineered, having opposing first positioning portion and second positioning portion and a high-rise portion connected between the first positioning portion and the second positioning portion and defining a high-rise space therebelow. Different heights of hard support plates can be selectively used with the protector body to fit different users, enabling the user to operate a computer keyboard or mouse comfortably for a long period of time without causing pain.

This application claims the priority benefit of Taiwan patent application number 099225646, filed on Dec. 31, 2010.

### BACKGROUND OF THE INVENTION

1. Field of the Invention

The present invention relates to a wrist protective device for protecting the user's wrist when the user is operating a computer keyboard or mouse and more particularly, to a wrist protector, which gives excellent support to the user's wrist without interfering with the movement of the user's hand in operating a computer mouse or keyboard and, which enables the user to operate a computer mouse or keyboard comfortably for a long period of time without causing any injury or pain.

2. Description of the Related Art

Following fast development of computer and multimedia technology, many different versatile electronic devices have entered our life and have become requisite tools in our daily life. Nowadays, a personal computer or notebook computer has become a tool popularly used by every individual in every family or office. Further, a computer generally provides a keyboard or mouse for data input or operation control. The keys or buttons of a computer mouse or keyboard are disposed at a relatively higher elevation than the surface on which the computer mouse or keyboard is rested. When operating the keys or buttons of a computer mouse or keyboard, the user must lift the palm of the hand. When lifting the palm of the hand, the palm gives a pressure to the wrist joint. Frequently giving a pressure to the wrist joint may cause wrist injuries or pain (carpal tunnel syndrome).

Further, wrist pads and mouse pads are commercially available for supporting the wrist of the user's hand when the user's hand is operating a computer keyboard or mouse. However, when using a wrist pad or mouse pad, it must be positively positioned on a flat surface at a fixed location. When a user is operating a computer keyboard or mouse, the user may have to lift the wrist from the wrist pad or mouse pad and move the hand or the mouse. When the user lifted the wrist from the wrist pad or mouse pad, the user cannot keep the wrist in one same posture. At this time, the wrist is not well supported or protected. Further, because the wrist pad or mouse pad is not movable with the hand, the user's operation in moving the computer mouse is limited to a certain range, causing operation inconvenience. In consequence, the applicability of conventional wrist pads and mouse pads is limited.

In order to settle the aforesaid problems, wrist protective devices are created. FIG. 10 illustrates a conventional wrist protective device, which comprises a cushion pad A having a side bar A1 located on each of two opposite lateral side walls thereof and a plurality of bottom holes A3 located on the bottom wall thereof, a wrist band A2 fastened to the side bars A1 and adapted for fastening the cushion pad A to the wrist of a person, a bottom plate C fastened to the bottom wall of the cushion pad A and a sliding structure B secured to the bottom side of the cushion pad A by the bottom plate C for enabling the cushion pad A to be moved on a flat surface. The bottom plate C has a plurality of countersunk holes C2 respectively located on the bottom corners thereof and affixed to the bottom wall of the cushion pad A with respective screws C3, and a plurality of ball holes C1 cut through the top and bottom sides thereof. The sliding structure B comprises a plurality of balls B1 respectively accommodated in the bottom holes A3 of the cushion pad A and partially protruding over the ball holes C1 of the bottom plate C, a plurality of compression springs B2 respectively mounted in the bottom holes A3 of the cushion pad A and stopped against the balls B1 to force the respective balls B1 toward the respective ball holes C1, and a plurality of rollers B3 respectively mounted in the bottom holes A3 of the cushion pad A and spaced around and stopped against the respective balls B1. This design of wrist protective device can be moved with the user's wrist on the top surface of a table to give support to the user's wrist. However, the cushion pad A is neither orthopedically engineered nor adjustable in height to fit different users (adults and children). A child using this design of wrist protective device for a long period of time may still cause pain or wrist injuries. Further, this design of wrist protective device has a complicated structure, increasing the manufacturing cost. Further, the cushion pad A has a large dimension. The matching precision among the cushion pad A, the sliding structure B and the bottom plate C is critical to assure structural stability. Further, because the cushion pad A is prepared from silicon rubber or a foam material, it will become aged or permanently deformed soon after a long use. When the cushion pad A is permanently deformed, it is not replaceable, and the whole wrist protective device must be thrown away.

### SUMMARY OF THE INVENTION

The present invention has been accomplished under the circumstances in view. It is one object of the present invention to provide a wrist protector, which gives support to the user's wrist without interfering with the movement of the user's hand in operating a computer mouse or keyboard. It is another object of the present invention to provide a wrist protector, which gives support to the user's wrist, enabling the user to operate a computer mouse or keyboard comfortably for a long period of time without causing any injury or pain.

To achieve these and other objects of the present invention, a wrist protector comprises a protector body having a flexible base member defining therein an accommodation chamber and a fastening structure adapted for fastening the flexible base member to a user's wrist, and a hard support plate detachably set in the accommodation chamber of the flexible base member to give support to the user's wrist. The hard support plate comprises opposing first positioning portion and second positioning portion and a high-rise portion connected between the first positioning portion and the second positioning portion and defining a high-rise space therebelow. When the user rests the wrist on a mouse pad or the top of a desk or computer table and operates a computer mouse or keyboard, the protector body and the support plate give support to the user's wrist without interfering with the movement of the user's hand in operating the computer mouse or keyboard. Therefore, the user can operate the computer mouse or keyboard comfortably for a long period of time without causing any injury or pain. Further, different heights of hard support plates can be selectively used with the protector body to fit different users, enabling the user to operate a computer keyboard or mouse comfortably for a long period of time without causing pain.

Further, the high-rise portion of the support plate has elongated slots cut through the opposing top and bottom sides thereof, providing a shock absorbing effect and facilitating ventilation.

Further, the support plate can be injection-molded from plastics, or made of a metal plate member by stamping. The simple structural design of the support plate facilitates the fabrication, saving much the tooling cost.

Further, the base member of the protector body has a protruding portion extended from one side thereof. The protruding portion has a beveled bottom edge for direct contact with the surface of a mouse pad or the top of a desk when the user is operating a computer keyboard or mouse. Subject to the design of the beveled bottom edge of the protruding portion, the lifting angle of the user's hand is limited to a predetermined range as the user's wrist is rested with the wrist protector on surface of the mouse pad or the top of the desk, avoiding wrist pain or stiffness due to a high pressure at the wrist angle.

Further, the flexible base member of the protector body has an insertion slot located on the bottom side thereof in communication with the accommodation space through which the hard support plate is inserted into the accommodation chamber and set therein. As the support plate is detachably mounted in the base member of the protector body, the user can remove the support plate from the protector body, facilitating washing of the protector body. Further, the protector body can be made in any of a variety of colors for selective use.

### BRIEF DESCRIPTION OF THE DRAWINGS

FIG. 1 is a perspective view of a wrist protector in accordance with the present invention.

FIG. 2 is an exploded view of the wrist protector in accordance with the present invention.

FIG. 3 is a side plain view, in an enlarged scale, of the support plate shown in FIG. 2.

FIG. 4 corresponds to FIG. 2 when viewed from another angle.

FIG. 5 is an exploded view in section of the wrist protector in accordance with the present invention.

FIG. 6 is a sectional assembly view of the wrist protector in accordance with the present invention.

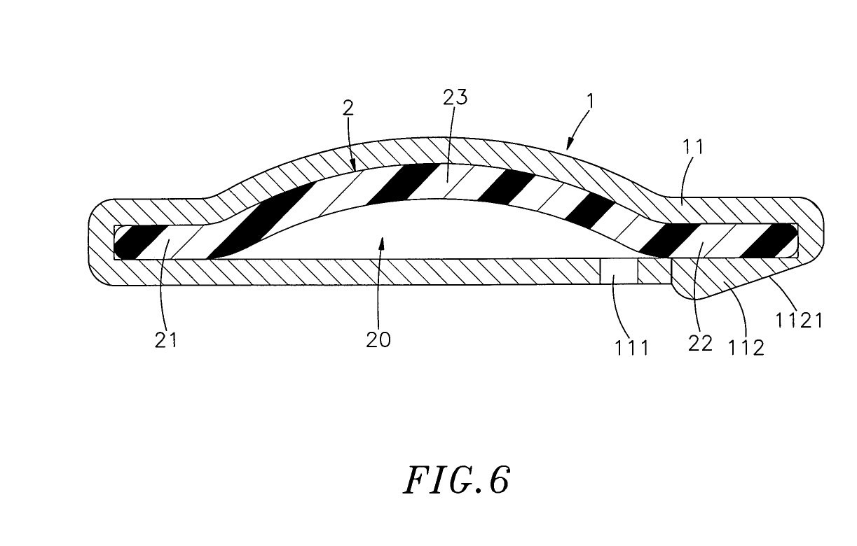

FIG. 7 is a schematic drawing illustrating a status of use of the wrist protector in accordance with the present invention.

FIG. 8 is an elevational view of an alternate form of the support plate for the wrist protector in accordance with the present invention.

FIG. 9 is a sectional side view of the support plate shown in FIG. 8.

FIG. 10 is an exploded view of a wrist protector according to the prior art.

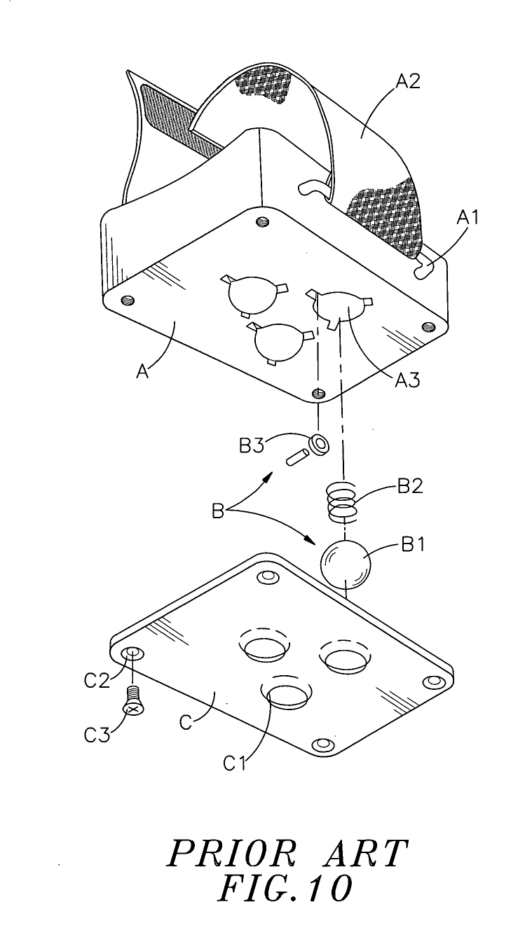

### DETAILED DESCRIPTION OF THE PREFERRED EMBODIMENT

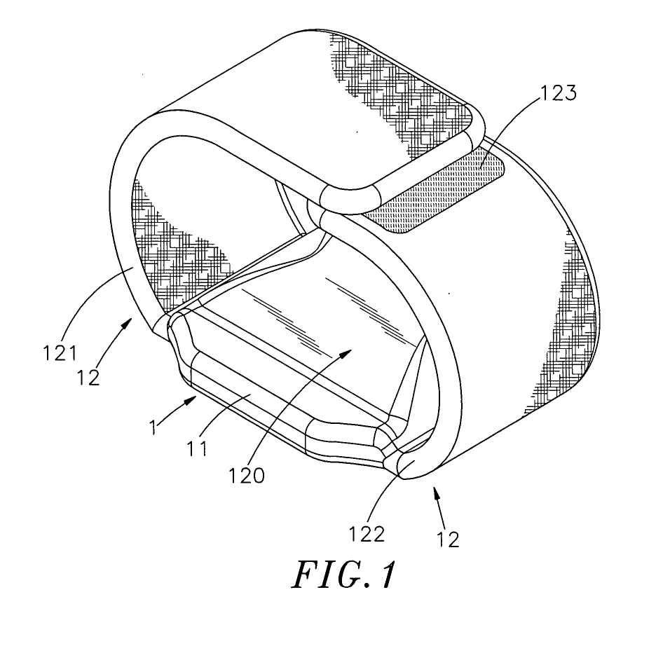

Referring to FIGS. 1˜4, a wrist protector in accordance with the present invention is shown comprising a protector body 1 and a support plate 2.

The protector body 1 includes a base member 11 and a fastening structure 12 adapted for fastening the base member 11 to the user's wrist. The base member 11 defines an accommodation chamber 10 and an insertion slot 111 on the bottom side thereof. Through the insertion slot 111, the support plate 2 is inserted into the accommodation chamber 10 and positively set therein. The fastening structure 12 includes a first wrist band 121, a second wrist band 122 respectively extended from two opposite lateral sides of the base member 11 and the fastening elements 123 respectively arranged on the distal end of the first wrist band 121 and the distal end of the second wrist band 122. By means of fastening the fastening element 123 at the first wrist band 121 and the fastening element 123 at the second wrist band 122 together, the first wrist band 121, the second wrist band 122 and the base member 11 form a loop, defining wrist-receiving space 120. Thus, the user can insert the hand through the wrist-receiving space 120 to let the protector body 1 be firmly secured to the user's wrist. Further, the first wrist band 121 and the second wrist band 122 can be fastened to the base member 11 with stitches or by an adhesive. Alternatively, the first wrist band 121, the second wrist band 122 and the base member 11 can be integrally made in a single piece. Further, the fastening elements 123 can be selected from hook and loop materials, snap fastener, magnetically attractive elements, etc.

The support plate 2 is set in the accommodation chamber 10 of the base member 11 and extends in a direction perpendicular to the extending direction of the first wrist band 121 and second wrist band 122 of the fastening structure 12 of the protector body 1, having opposing first positioning portion 21 and second positioning portion 22 and a high-rise portion 23 connected between the first positioning portion 21 and the second positioning portion 22. According to this embodiment, the high-rise portion 23 is smoothly arched, defining a high-rise space 20 therebelow between the first positioning portion 21 and the second positioning portion 22.

Further, the aforesaid protector body 1 can be made of a woven fabric, synthetic fibers, leather, silicon rubber or any other suitable elastic or flexible material. The first wrist band 121 and second wrist band 122 of the fastening structure 12 of the protector body 1 can be elastic bands or flat ropes, and detachably fastened together by means of the fastening elements 123 (hook and loop materials, snap fastener or magnetically attractive elements). Alternatively, the fastening structure 12 of the protector body 1 can be a single-piece elastic band or rubber band for directly fastening to the user's wrist positively and comfortably. Further, the support plate 2 can be made of plastics, wood or metal, having a thickness t=3˜5 mm, a length D=75˜85 mm, and a height H=11˜16 mm, wherein the first positioning portion 21 has a length d1=8˜13 mm; the second positioning portion 22 has a length d2=12˜17 mm; the high-rise portion 23 has a radius of curvature R=35˜55 mm.

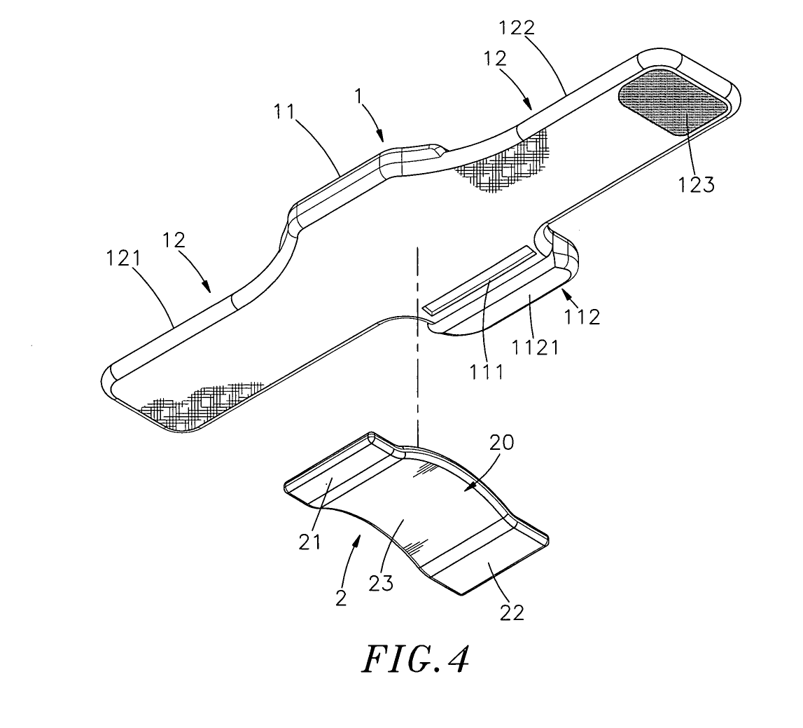

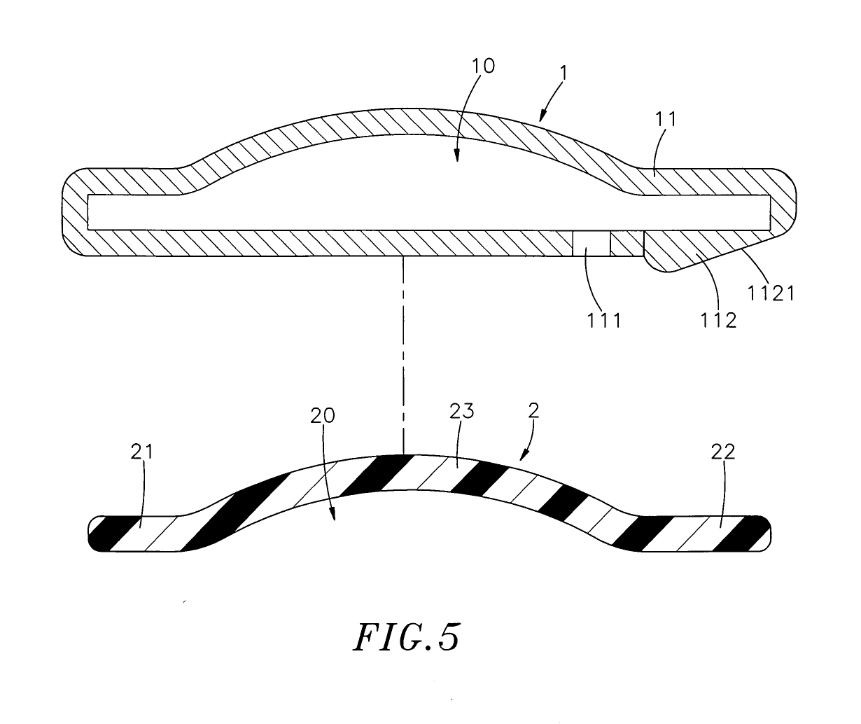

Referring to FIGS. 5˜7 and FIG. 4 again, before using the wrist protector, insert, the support plate 2 through the insertion slot 111 of the base member 11 of the protector body 1 into the accommodation chamber 10 and positively set the support plate 2 in the accommodation chamber 10 in a perpendicular manner relative to the first wrist band 121 and the second wrist band 122. At this time, the support plate 2 is received inside base member 11 and kept from sight. As the support plate 2 is detachably mounted in the base member 11 of the protector body 1, the user can remove the support plate 2 from the protector body 1, facilitating washing of the protector body 1. Further, the protector body 1 can be made in any of a variety of colors for selective use.

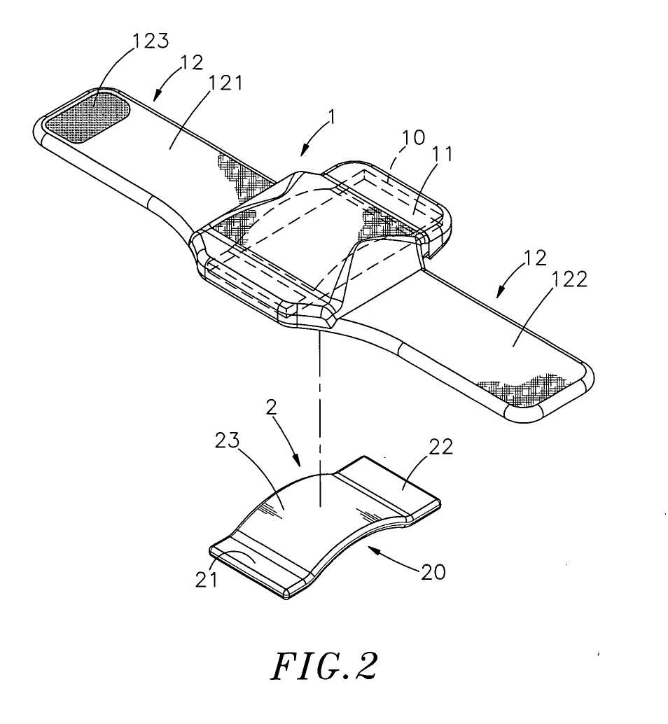

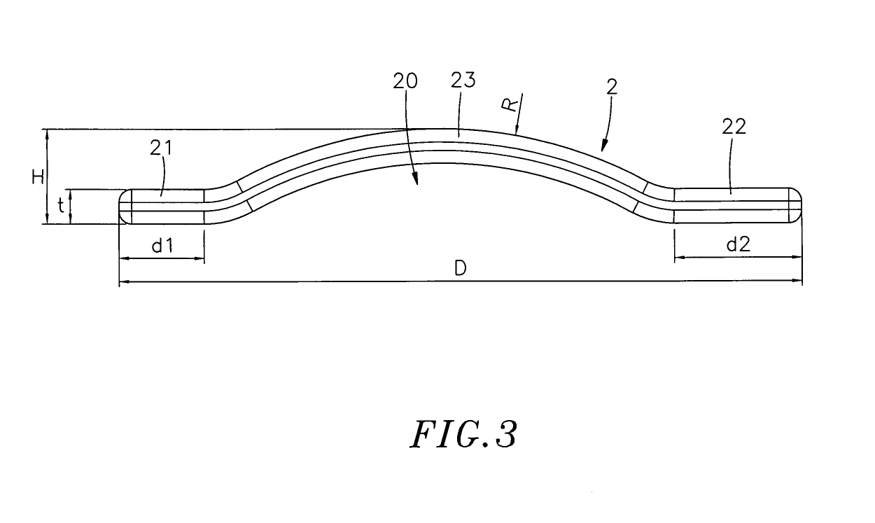

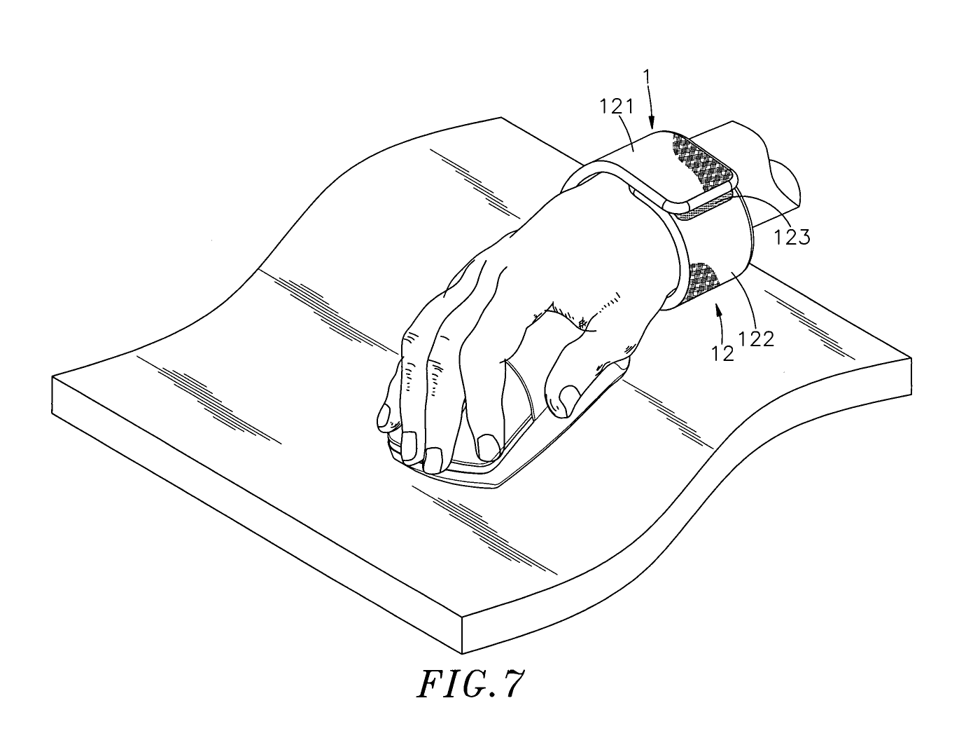

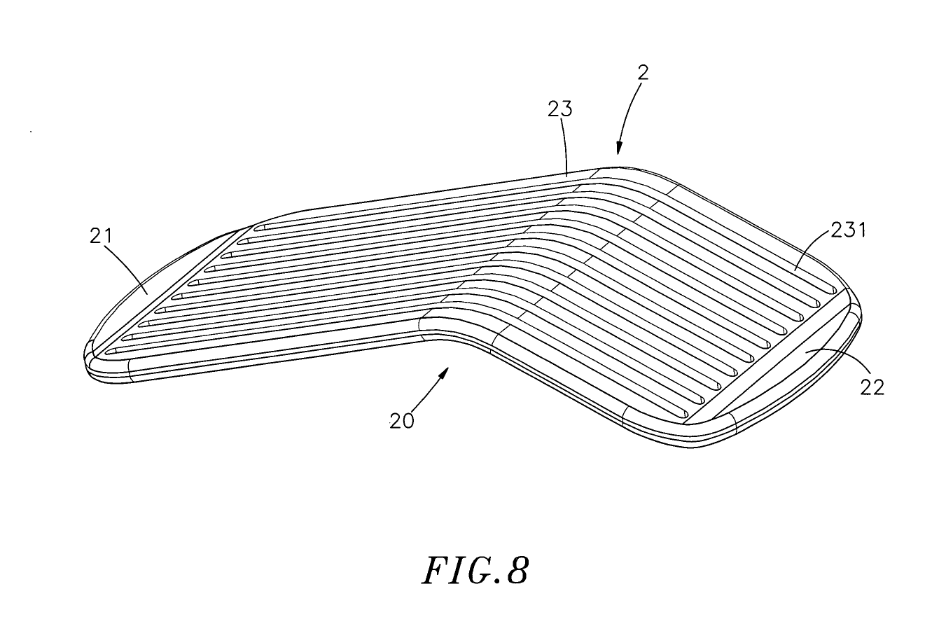

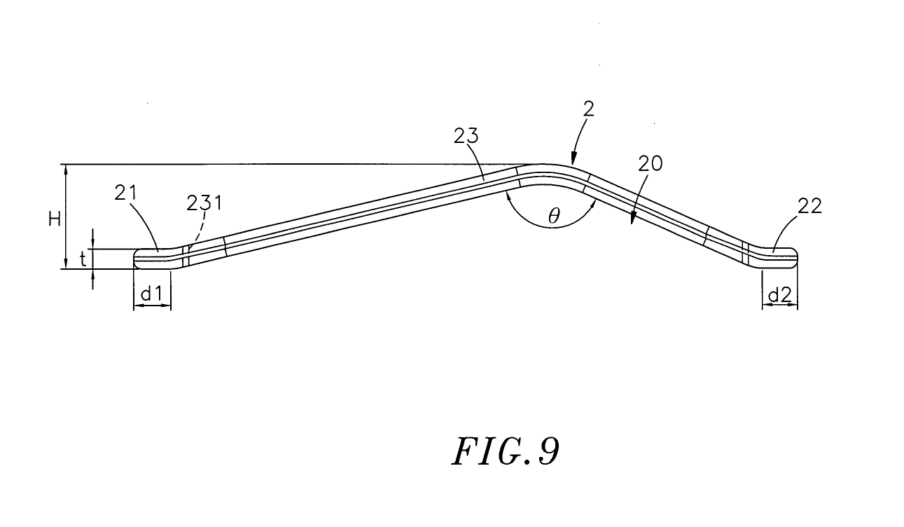

Referring to FIGS. 8 and 9 and FIGS. 2, 3 and 7 again, during application of the wrist protector, attach the user's wrist to the top side of the base member 11 of the protector body 1, and then wind the first wrist band 121 and second wrist band 122 of the fastening structure 12 of the protector body 1 round the user's wrist and fasten the fastening element 123 at the first wrist band 121 and the fastening element 123 at the second wrist band 122 together to secure the protector body 1 to the user's wrist and to let the user's wrist be received in the wrist-receiving space 120. Thus, when the user rests the wrist on a mouse pad or the top of a desk or computer table and operates a computer mouse or keyboard, the protector body 1 and the support plate 2 give support to the user's wrist without interfering with the movement of the user's hand in operating the computer mouse or keyboard. Therefore, the user can operate the computer mouse or keyboard comfortably for a long period of time without causing any injury or pain. Further, different heights of support plates 2 may be provided for selective use.

Further, as shown in FIGS. 4˜6, the base member 11 of the protector body 1 further has a protruding portion 112 extended from one side thereof adjacent to the insertion slot 111. The protruding portion 112 has a beveled bottom edge 1121 for direct contact with the surface of the mouse pad or the top of the desk. Subject to the design of the beveled bottom edge 1121 of the protruding portion 112, the lifting angle of the user's hand is limited to a predetermined range as the user's wrist is rested with the wrist protector on surface of the mouse pad or the top of the desk, avoiding wrist pain or stiffness due to a high pressure at the wrist angle.

In actual practice, the support plate 2 preferably has a thickness t=4 mm, a length D=80 mm, and a height H=11, 13 or 16 mm, wherein the first positioning portion 21 has a length d1=10 mm; the second positioning portion 22 has a length d2=15 mm; the high-rise portion 23 has a radius of curvature R=41.51 mm or 50.45 mm. In an alternate form of the present invention, the high-rise portion 23 is double-beveled, defining an obtuse angle between the first positioning portion 21 and the second positioning portion 22 and having a plurality of elongated slots 231 cut through the opposing top and bottom walls thereof and extending in a parallel manner between the first positioning portion 21 and the second positioning portion 22. Further, the corners of the support plate 2 are chamfered. Subject to the design of the elongated slots 231, the high-rise portion 23 of the support plate 2 can lessen or absorb shocks and facilitates ventilation. Further, the support plate 2 of this alternate form can be injection-molded from a plastic material, or made of a metal plate member by means of a stamping technique. Thus, the fabrication of the support plate 2 is easy and inexpensive, saving much the cost. Further, a series of support plates 2 of different heights can be provided for use with the protector body 1 selectively to fit different requirement. The mounting design of the invention enables the user to replace the support plate 2 conveniently and rapidly.

The above described embodiments are simply exemplars of the invention but not intended for use as limitations of the invention. In general, the invention provides a wrist protector comprising a protector body 1, which includes a base member 11 and a fastening structure 12 adapted for fastening the base member 11 to the user's wrist, and a support plate 2 detachably mounted in the base member 11 of the protector body 1. When the user rests the wrist on a mouse pad or the top of a desk or computer table and operates a computer mouse or keyboard, the protector body 1 and the support plate 2 give support to the user's wrist without interfering with the movement of the user's hand in operating the computer mouse or keyboard. Thus, the user can operate the computer mouse or keyboard comfortably for a long period of time without causing any injury or pain. Further, the simple structural design of the support plate 2 facilitates the fabrication, saving much the tooling cost. Further, different heights of support plates 2 may be provided for selective use with the protector body 1.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. A wrist protector, comprising:
a protector body comprising a flexible base member and a fastening structure adapted for fastening said flexible base member to a user's wrist, said flexible base member defining therein an accommodation chamber; and
a hard support plate set in said accommodation chamber of said flexible base member to give support to the user's wrist, said hard support plate comprising opposing first positioning portion and second positioning portion and a high-rise portion connected between said first positioning portion and said second positioning portion and defining a high-rise space therebelow between said first positioning portion and said second positioning portion.

2. The wrist protector as claimed in claim 1, wherein said flexible base member comprises an insertion slot located on a bottom side thereof in communication with said accommodation space through which said hard support plate is inserted into said accommodation chamber and set therein.

3. The wrist protector as claimed in claim 1, wherein said fastening structure of said protector body comprises a first wrist band, a second wrist band respectively extended from two opposite lateral sides of said flexible base member and a set of fastening elements respectively arranged on the distal end of said first wrist band and the distal end of said second wrist band remote from said flexible base member and adapted for joining the distal ends of said first wrist band and said second wrist band together.

4. The wrist protector as claimed in claim 3, wherein said set of fastening elements is prepared from hook and loop materials.

5. The wrist protector as claimed in claim 3, wherein said set of fastening elements is a snap fastener.

6. The wrist protector as claimed in claim 3, wherein said set of fastening elements is a set of magnetically attractive elements.

7. The wrist protector as claimed in claim 3, wherein said high-rise portion of said hard support plate is smoothly arched and extending in a perpendicular manner relative to the extending direction of said fastening structure.

8. The wrist protector as claimed in claim 3, wherein said high-rise portion of said hard support plate is double-beveled and extending in a perpendicular manner relative to the extending direction of said fastening structure.

9. The wrist protector as claimed in claim 1, wherein said hard support plate has a thickness about 3˜5 mm, a length about 75˜85 mm, and a height about 11˜16 mm; said first positioning portion has a length about 8˜13 mm; said second positioning portion has a length about 12˜17 mm; said high-rise portion has a radius of curvature about 35˜55 mm.

10. The wrist protector as claimed in claim 1, wherein said hard support plate has a thickness about 4 mm, a length about 80 mm, and a height selected from one of 11 mm, 13 mm and 16 mm; said first positioning portion has a length about 10 mm; said second positioning portion has a length about 15 mm; said high-rise portion has a radius of curvature selected from one of 41.51 mm and 50.45 mm.

11. The wrist protector as claimed in claim 1, wherein said high-rise portion of said hard support plate is double-beveled, defining an obtuse angle between said first positioning portion and said second positioning portion.

12. The wrist protector as claimed in claim 1, wherein said high-rise portion of said hard support plate has a plurality of elongated slots cut through opposing top and bottom sides thereof.

13. The wrist protector as claimed in claim 1, wherein said protector body is selected from the material group of woven fabric, synthetic fibers, leather and silicon rubber.

14. The wrist protector as claimed in claim 1, wherein said hard support plate is selected from the material group of hard plastics, wood and metal.
